(19) United States
(12) Patent Application Publication
(10) Pub. No.: US 2007/0269854 A1
(43) Pub. Date: Nov. 22, 2007

(54) Method and Device for Measuring an Enzymatic Activity in a Body Fluid

(75) Inventors: Jean-Maxime Nigretto, I'Isle-Adam (FR); Guillaume Lamblin, Rouvray (FR); Marielle Paris, Rome (IT); Norbert Benattar, St Jean de Vedas (FR)

Correspondence Address: OBLON, SPIVAK, MCCLELLAND MAIER & NEUSTADT, P.C., 1940 DUKE STREET, ALEXANDRIA, VA, 22314, US

(73) Assignees: BIOPEP, Mauguio (FR); UNIVERSITE DE CERGY PONTOISE, Cergy Pontoise Cedex (FR)

(21) Appl. No.: 11/573,797

(22) PCT Filed: Aug. 5, 2005
(86) PCT No.: PCT/FR05/02031

§ 371 Date: Apr. 17, 2007

(30) Foreign Application Priority Data
Aug. 18, 2004 (FR) 0408960

(51) Int. Cl. C12M 1/34; G01N 33/48

(52) U.S. Cl. 435/40.51; 435/287.9; 435/40.5

(57) ABSTRACT

The invention concerns a method and a device for measuring an enzymatic activity in a body fluid. The method comprises the following steps: a) providing a support having a conductive surface whereon is immobilized a substrate specific of the enzymatic activity to be measured, the substrate comprising an electroactive residue; b) reacting a sample of the body fluid with the substrate so as to bring about an enzymatic reaction generating an electroactive product; c) detecting the amount of electroactive product by amperometric measurement using a support in contact with the medium containing the enzyme. The invention is useful in particular for measuring enzymatic activities related to blood coagulation.

The invention relates to the field of biological analyses and more particularly to a method and device for measuring an enzymatic activity in a body fluid, notably in whole blood.

The determination of given enzymatic activities in certain body fluids takes on particular importance for the diagnosis and treatment of certain complaints.

This is true in particular of the measurement of various enzymatic activities linked with the coagulation of whole blood. In fact, the measurement of these enzymatic activities presents particular problems when the body fluid in question is cloudy and/or coloured and can therefore give rise to difficulties in optical methods of analysis.

In the case of whole blood, which is a cloudy, coloured fluid, it is certainly possible to extract the plasma for subsequent analysis However, the preparation of the plasma requires a certain number of pre-analytical steps and is moreover a source of potential errors.

A method of titrating proteases and anti-proteases in clotting systems is already known from U.S. Pat. No. 4,304,853 in the names of Nigretto and Jozefowicz.

This patent describes, more particularly, a method of titrating proteases and anti-proteases, and particularly proteases and anti-proteases in clotting systems and in complement. This process consists in reacting the enzyme which is to be titrated with a substrate which is electrochemically neutral but which, after hydrolysis with the enzyme, yields a hydrolysis product capable of being oxidised or reduced electrochemically within the electroactivity range of the system, this product then being measured by amperometry at a pH of between 2 and 10.

The titration of the enzyme is carried out here by a particular electrical measurement which is defined in the patent.

A particular example of the substrate used is benzoyl-D,L-arginyl-p-aminodiphenylamide chloride. The enzymatic reaction thus makes it possible to free the corresponding amine, namely p-aminodiphenylamine, also known as pADA, for short.

However, this known method of titrating and measuring coagulation proteases and anti-proteases is carried out only in a homogeneous medium, i.e. in solution.

Patent EP 1 031 830 in the name of the company ASULAB chiefly picks up on the principle of an electrochemical detector from U.S. Pat. No. 4,304,853 mentioned above.

ASULAB propose measuring the prothrombin time by measuring the electrical activity generated by an enzymatic reaction with an electrogenic substrate formed by a charged group which can be cut rom its chain by thrombin. The surface to which the substrate is fixed is a layer of platinum or silver.

As in U.S. Pat. No. 4,304,853 the reaction is carried out in a homogeneous medium, i.e. in solution.

The publication WO 01/63271 in the name of ROCHE DIAGNOSTICS takes up the idea of using an electrochemical measurement to measure coagulation. This patent describes a reaction in a homogeneous medium and uses a reagent which is not fixed to a solid support to detect thrombin. The device described comprises measuring electrodes made of palladium. However, this publication does not explicitly mention measuring clotting factors.

U.S. Pat. No. 6,495,336 of Messrs PENTAPHARM describes products which allow electrochemical measurement of the activity of the proteins of coagulation, and more precisely the thrombin activity. The patent claims a reaction in a homogeneous medium in the presence of gold or platinum electrodes, the measuring principle being related to the technology described in U.S. Pat. No. 4,304,853 mentioned above.

The publication WO 01/36666 by the company I-STAT relates to an apparatus for measuring coagulation using an electrochemical sensor. The apparatus uses a reagent to trigger a clotting reaction. Sensors then directly measure the enzymatic activity responsible for the coagulation.

Here again, the reaction described in the patent is a reaction in a homogeneous medium. i.e. in solution.

The invention sets out, in particular, to improve the situation by proposing a new method and a new device for measuring an enzymatic activity in a body fluid which avoids the drawbacks of the prior art.

It is a particular aim of the invention to provide a method and device of this kind which are suitable for measuring different enzymatic activities in different types of body fluids, and particularly in body fluids which are cloudy and/or coloured, as is the case with whole blood.

A further aim of the invention is to provide a method and device of this kind which are suitable for measuring enzymatic activities present in the blood and especially enzymatic activities linked with blood clotting.

A further aim of the invention is to provide a method and device of this kind which make it possible to carry out a reaction in different types of medium, not only in homogeneous media but also in heterogeneous media

The invention relates more particularly to a method of measuring an enzymatic activity in a body fluid, which comprises the following steps:

a) providing a support having a conductive surface whereon is immobilised a substrate specific to the enzymatic activity to be measured, the substrate comprising an electroactive residue;

b) reacting a sample of the body fluid with the substrate so as to bring about a enzymatic reaction generating an electroactive product shown up by enzymatic hydrolysis;

c) detecting the amount of electroactive product by amperometric measurement using the support in contact with the medium containing the enzyme.

The invention thus makes it possible to detect an electroactive product acting as a marker, which car be freed from the support or remain attached to the support the quantity of which provides a direct measurement of the enzymatic activity by amperometry

In the present application, the terms mentioned below have the meanings specified:
amperometric: measurement of a current generated by a potential excitation, either momentary or dependent on time;
substrate: molecule recognised and converted by the enzyme;
   marker: the product of an enzymatic reaction which is an electroactive group that can be detected by an electrode;
electroactive: capable of being oxidised or reduced;
diffusing: moving under the influence of a concentration gradient;
reduced volume: micro- or submicrovolumetric (typically less than 100 microlitres);
working electrode: conductor, seat of the electrochemical or capacitive reaction which provides the measurement.

In a preferred embodiment of the invention the substrate is deposited in the form of at least one monomolecular layer of molecules corresponding to one of the following general formulae I and II:
S-Cₙ-(E)-P-X (I)
S-Cₙ-(E)-X-P (II)
wherein:

S denotes a chemical group capable of forming a bond with the conductive surface,

Cₙ denotes an aliphatic methylene chain consisting of n carbon atoms,

E denotes a non-electroactive hydrophilic spacer of the polyethylene glycol type, which may optionally be present to complete the chain Cₙ,

P denotes a polypeptide sequence specific to the enzymatic activity which is to be measured, and

X denotes an electroactive residue capable of being oxidised or reduced within a potential range which is accessible in the medium.

In formulae I and II, n is a whole number between 9 and 18.

Thus, the conductive surface of the support is modified by depositing a monomolecular layer or monomolecular strata of molecules each corresponding to one or other of general for formulae I and II specified above. These molecules have the property of self-assembling spontaneously on the conductive surface. They are also known as SAM, an abbreviation for the English term “SELF-ASSEMBLED MONOLAYERS”.

The hydrophilic spacer E is optional. Its insertion in the sequence of formula I or formula II is useful for increasing the surface hydrophilicity of the substrate, on the solution side, thus reducing any phenomena of short-distance repulsion, irreversible adsorption or denaturing of the enzyme as it approaches the surface.

In a preferred embodiment, in formulae I and II, S denotes a thiol group, Cₙ denotes a hydrophobic carbon chain comprising n hydrocarbon members, particularly methylene and carrying a hydrophilic spacer E, P denotes a peptide sequence with at least two amino acids of a substrate specific to the enzymatic activity which is to be measured, and X is an aromatic amine, particularly para-aminodiphenylamide.

S thus denotes a thiol group, also known as mercapto, connected to a carbon chain comprising n methylene members (n is typically of the order of 10) and carrying a hydrophilic spacer E. This spacer is preferably of the ethylene glycol type.

P denotes a peptide sequence having at least two amino acids, typically between 2 and 5 amino acids. This peptide is chosen for its specificity for the enzyme which is to be measured, for example thrombin, when measuring an activity linked to blood clotting.

X is advantageously an aromatic amine, particularly para-aminodiphenylamide, Enzymatic hydrolysis shows up the corresponding amine, namely p-aminodiphenylamine. This amine was chosen for its notable hydrophobicity. This property promotes the sensitivity of detection inasmuch as it increases the coefficient of division: concentration of the free amine in the substrate/concentration of the free amine in solution. Furthermore, the amine is oxidised at low potentials, which makes it easy to detect in the presence of interfering agents which are also oxidisable, such as ascorbic acid.

As already pointed out, the invention is of particular interest where the body fluid is a cloudy and/or coloured fluid, as in the case with whole blood.

In this respect, the enzymatic activity to be measured may be that of a clotting factor or complement of whole blood, particularly a clotting factor such as thrombin. In connection with coagulation, the enzymatic activity may be used to titrate an inhibitor or an endogenous or exogenous cofactor of blood coagulation, given that the factors and inhibitors may be physiological or exogenous. A particular example of an exogenous inhibitor is heparin, a substance given to patients to inhibit blood clotting.

For the reaction in step b) a drop of the sample, particularly whole blood, may be placed directly on the support on which the substrate is immobilised. Under these conditions, depositing a small amount of fluid, e.g. a single drop of blood, on the support and the substrate, constituting an active sensor, the measuring site, enables fast and direct detection of the enzymatic activities under investigation.

With regard to measurements linked with the coagulation of whole blood, the invention does away with all the conventional pre-analytical steps needed to prepare the plasma, thus saving time and eliminating potential sources of error.

Thus, in this particular application, the invention makes it possible to measure an enzymatic activity from a small sample of body fluid.

When the sample of fluid, for example the drop of blood, comes into contact with the substrate, the enzyme which is to be quantified reacts with the immobilised substrate. This enzymatic reaction generates an electroactive product detected by the measuring electrode integrated in the sensor. The amplitude of the signal measured is proportional to the quantity of product formed during the reaction, thus providing a measurement of the activity of the clotting factor.

This measuring system can be used directly by the patient or by a hospital staff member.

One of the advantages of the invention is that it allows very rapid measurement of enzymatic activities, e.g. clotting factors or clotting inhibitors. The invention is thus of particular value in emergency departments as a rapid diagnostic tool, or in the patient's home, for the purpose of measuring the progress of a particular enzymatic activity.

The conductive surface is advantageously a layer of a metal capable of immobilising the covalent molecules I or II, especially gold. Preferably, it is a layer of gold deposited on a silicon-based support.

The detection step is carried out with electrodes which typically comprise a working electrode, a counter-electrode and optionally a reference electrode. The working electrode advantageously consists of the support itself. The counter-electrode and the reference electrode may be separate; however, it is advantageous if the counter-electrode and the reference electrode are one and the same, the counter-electrode then serving as a potential reference.

The detection step advantageously comprises measuring the amplitude of a signal proportional to the quantity of electroactive product shown up during the enzymatic reaction.

In another aspect the invention relates to a device for measuring an enzymatic activity in a body fluid, which can be used to carry out the method defined hereinbefore.

This device essentially comprises:
a support having a conductive surface;
a substrate specific to the enzymatic activity which is to be measured and comprising an electroactive residue, the substrate being immobilised on the conductive surface;
means for detection by electrochemical, particularly amperometric measurement.

In this device the substrate is advantageously deposited in the form of at least one monomolecular layer of molecules corresponding to one or other of general formulae I and II as mentioned hereinbefore. Consequently, the features defined hereinbefore for the method also apply to the device.

In one embodiment of the invention the amperometric detection means comprise a potentiostat capable of delivering, between the working electrodes and the reference electrode, a periodic potential difference which is adjustable to the range of detection of the marker.

In the description that follows, provided by way of example reference is made to the attached drawings, wherein.

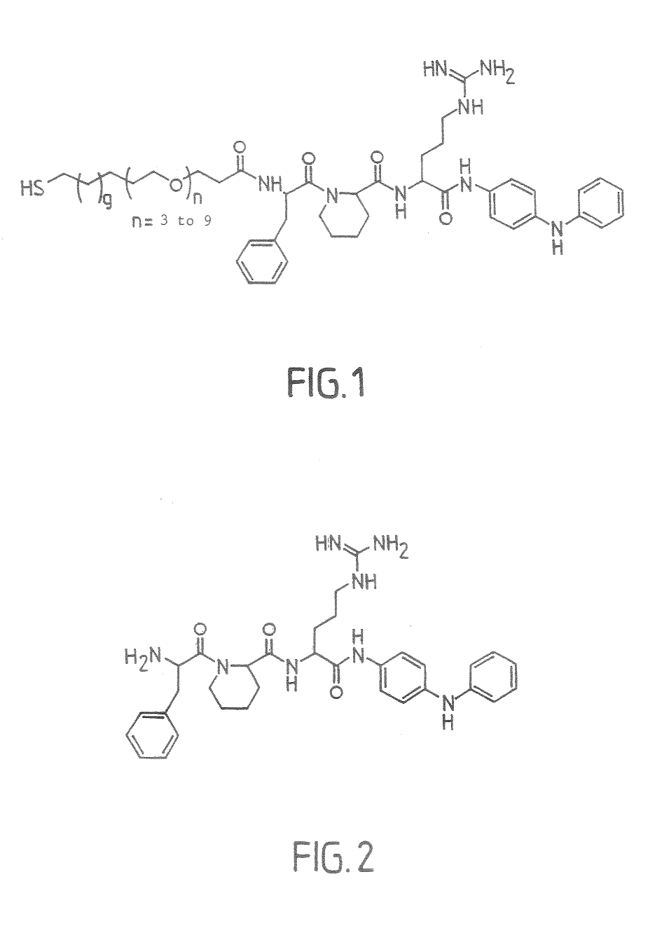

FIG. 1 is the developed formula of an example of a substrate according to the invention;

FIG. 2 shows the developed formula of the peptide sequence of the substrate of FIG. 1;

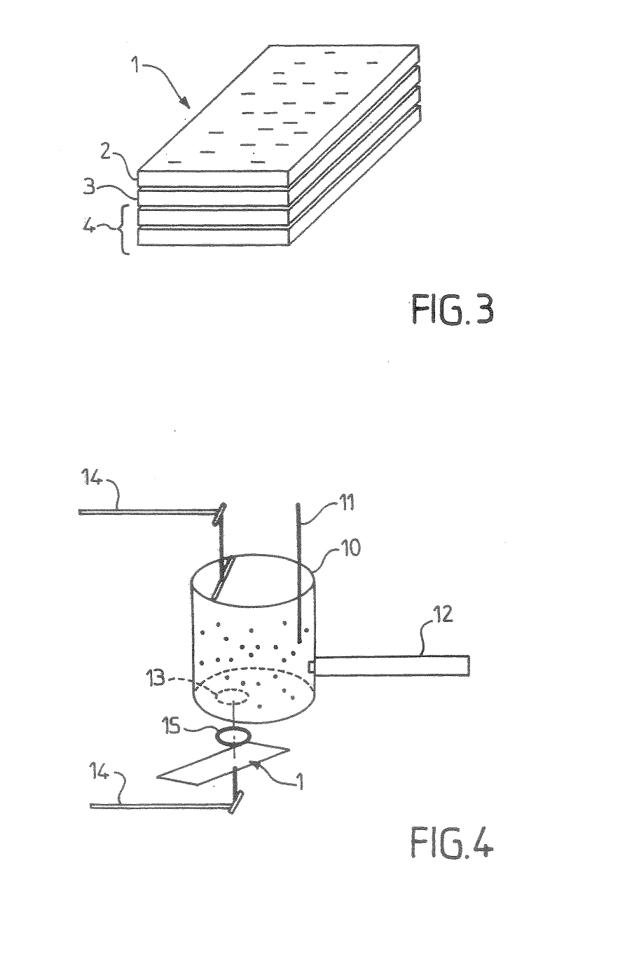

FIG. 3 is a perspective view of a electrode according to the invention;

FIG. 4 is a perspective view of a measuring cell used in the invention;

FIG. 5 shows the amperometric detection of p-aminodiphenylamine (pADA) in solution by means of a surface of gold modified with a monolayer of the substrate the formula of which is shown in FIG. 1;

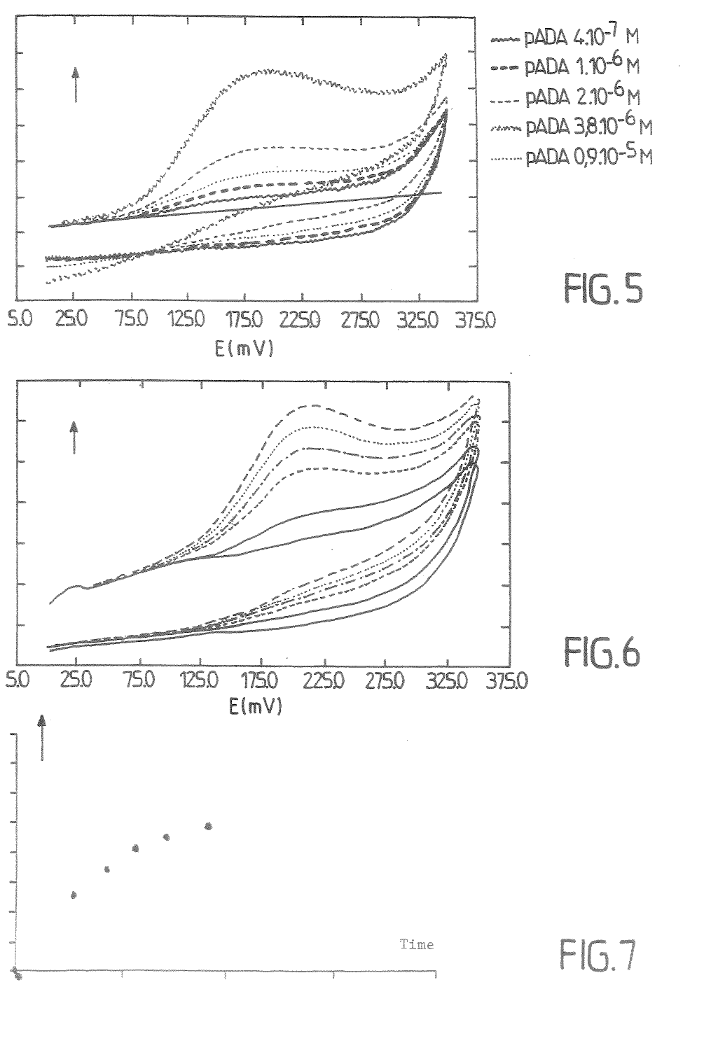

FIG. 6 shows the progressive variation in a voltametric current recorded during hydrolysis kinetics in the presence of 80 mU/ml of trypsin; and

FIG. 7 is a graph showing the development of the currents in FIG. 6 as a function of time.

The invention will be described by means of the example that follows:

### EXAMPLE

The Example described relates to the titration of trypsin for the purpose of testing the method and device according to the invention. Trypsin is a protease of the same kind as the enzymes of haemostasis. Trypsin is a protease of the same kind as the enzymes of haemostasis.

The system used comprises a conductive surface, preferably consisting of a noble metal such as gold, modified by the depositing of a monomolecular layer or monomolecular strata of molecules corresponding to one or other of general formulae I and II as mentioned hereinbefore.

In these formulae, S is a thiol group (also Known as mercapto), Cₙ is a carbon chain having 11 methylene chain members, E is a hydrophilic spacer of the hexa-ethylene glycol type, P is a tripeptide sequence of a substrate hydrolysed by trypsin (the formula of which is shown in FIG. 2) and X is para-aminodiphenylamide.

The amine which will be released by enzymatic hydrolysis, namely p-aminodiphenylamine, was chosen for its notable hydrophobicity, as mentioned previously. This amine is oxidised at low potentials, which makes it easy to detect in the presence of interfering agents which are also oxidisable, such as ascorbic acid.

The preparation of the surface will now be described. This surface is a rectangular bar 1 (see FIG. 3) having a layer of gold 2 on its surface which is deposited on a layer of titanium 3, which is in turn deposited on a silicon-based bar 4.

Typically, the layer of gold is 3000 Angstroms thick, the layer of titanium is 300 Angstroms thick and the silicon bar is 500 microns thick. The whole is annealed for 20 minutes at 250° C. under vacuum The process is carried out using the normal techniques for producing information carriers in microprocessing or microelectronics. However, it is necessary to pretreat the gold in order to optimise the formation of a self-assembled monolayer having the required properties. The surface, which is handled with non-paraffinated protective gloves, is carefully degreased using acetone and then ethanol, then rinsed with ultra pure water, with ethanol again and finally dried under a nitrogen current.

It is then used as a working electrode in a conventional electrochemical assembly having three electrodes (working electrode, counter-electrode and reference electrode, and immersed in a solution of pure water containing 0.5% by volume of sulphuric acid.

The pretreatment operations will now be described. Earlier methods of preparing surfaces for the formation of self-assembled monolayers (SAM) use either electrochemistry or physical methods in vacuo. It has been possible to obtain a reproducible and controlled surface state using the electrochemical process. This comprises subjecting the bar to a number of cyclovoltamograms of between 0.0 and 1.5 V/reference Ag/AgCl at a sweep speed of 0.1 V/s which is sufficient to achieve stabilisation of the response. This takes around 30 cycles. The i/E cur e (current/potential) then shows up a series of peaks attributed to the formation of gold oxides in the zone 1.2 to 1.5 V, followed, after inversion of the potential, by a narrow peak for the reduction of these oxides towards 0.8 V. The integration of the reduction peak makes it possible to evaluate the effective surface of the electrode and hence, by comparison with the geometric surface, its roughness. The electrode is removed from the solution at a final potential of 0.0 V leaving the gold free from oxide which would be prejudicial to the formation of SAM, as described in the literature.

The experiments are carried out with an EC+G Princeton Applied Research potentiostat, model 273A, equipped with ECHEM or Power Suite EG & G software.

The depositing of the assembled monolayer (SAM) will now be described. After vigorous rinsing with ultra pure water, the bar is left with stirring at ambient temperature for at least 12 hours in 10 ml of ethanol containing a homogeneous mixture of electrogenic substrate and mercapto-hexanol coupled with a C₃ polyethylene glycol chain in a proportion of between 0.1 and 10% respectively, calculated to give a total concentration of 0.1 mM. The mercapto-hexanol acts as a diluent of the substrate in FIG. 1 on the surface. Negative preliminary results obtained with an SAM free from diluting thiol lead one to believe that the surface dilution of the electrogenic substrate results in better accessibility of the enzyme to the substrate and makes the local physico-chemical conditions more favourable to achieving enzymatic hydrolysis. Other studies carried out in the laboratory and the information collected in the literature for molecules of comparable structure indicate that a modification time of 12 hours is sufficient to achieve surface saturation by the SAM. The density of the immobilised SAM was evaluated using the quartz micro-balance method (QCM). It reaches about 4 H 10¹⁰ mol/cm², in agreement with the published data relating to analogues.

After deposition, the SAM may be characterised by various methods:
electrochemical desorption: this method consists in subjecting the modified surface to a sweep of reducing potential from 0 to −1.4 V in a 0.5 M KOH medium at 0.05 V/s. Under these conditions, each set of bonds (Au—S) present on the surface undergoes electrochemical reduction which leads to the desorption of the organic molecule. This reaction uses up electrons. Therefore a desorption peak appears, the potential of which is characteristic of each thiol Integration of the peak gives the surface density of the thiol.
   electrochemical oxidation: the electrogenic substrate immobilised has an oxidation signal beyond 0.4 V corresponding to the oxidation of X coupled to the substrate. The integration of the response gives the surface density of the thiol.
   weight: an SAM deposited on gold supported by a quartz crystal lends itself to weighing by the QCM technique.
   permeation using reversible redox markers. The kinetics of the progressive modification of gold by the SAM are monitored by the development of the cyclovoltametric signal of known simple redox pairs to give a reversible or quasi-reversible signal, such as the pair Fe(CN)₆^{3-/4-} (hexacyanoferrate III/II) or Ru(CN)₆^{3+/2+} (hexacyanoruthenate III/II). On non-modified gold the reversibility of the signals is characterised by a separation of the anodic and cathodic peaks of oxido-reduction of the order of 65 to 80 mV in a Tris 0.15 M/KCl 0.5 M pH 7.4 medium. As the SAM is formed, the intensity of the anodic and cathodic currents weakens until an exponential variation is obtained. By recourse to different theories (Amatore-Teyssier-Savéant or Finklea theories) it is possible to evaluate the density of the defects responsible for the persistence of the voltametric oxido-reduction signal of the markers.
   capacitance: the residual current obtained in the presence of an SAM lowers the residual current of the bare surface as a result of the capacity of the self-assembled molecular layer. The typical capacity of the double bare layer, of the order of 20 μF/cm² on bare gold, falls to 2 to 6 μF/cm² after modification. In the zone from 0.0 to 0.35 V the capacity of the SAM of the support having the formula in FIG. 1 is not very sensitive to the potential.

The electrochemical analysis is carried out using a device as shown in FIG. 4. The electrochemical analysis of the activity of trypsin is carried out in a cylindrical container 10 made of polytetrafluoroethylene containing 5 ml of Tris 0.15 M/KCl 0.5 M buffer at pH 7.4 using the bar 1 as a working electrode, a platinum wire 11 as a counter-electrode and an Ag/AgCl electrode 12 as the reference electrode inserted laterally in the container, leaving only the endpiece in contact with the solution in order to minimise the adsorption of protein on the glass. It is also possible to use only two electrodes: the working electrode and a silver wire which then acts as a pseudo-reference, provided that care is taken to separate the two electrodes sufficiently so that the phenomena occurring on the counter-electrode do not interfere with those occurring on the working electrode.

The surface of the bar, placed horizontally under the container, is accessible to the solution through a circular port 13 formed through the base of the container. A leaktight seal is provided by mechanical pressure exerted by a mechanical clamping system 14 and by the interposition of the toroidal joint 15 made of solvent-resistant polymer between the container and the bar.

The electrochemical signal will now be described.

The response of the modified surface in the presence of trypsin is obtained by applying a series of triangular voltametric signals between 0.0 and 0.35 V at a speed of 0.1 V/s and periodically at a rate which depends on the enzyme concentration (between 10 and 300 s). The maximum potential should not exceed 0.35 V, so as to avoid the danger of oxidising the non-hydrolysed substrate. This limit is also designed not to affect the integrity of the SAM. The background noise of the system is first recorded in the sample solution of trypsin. This gives superimposed voltamograms the currents of which will be subtracted from those obtained in the presence of enzyme. Then, once the enzyme has been added the first voltamogram of the kinetics is traced giving the current at t=0.

The enzyme needed to obtain a final concentration of 80 mU BAEE/ml (BASE=benzoyl arginine ethyl ester) is added at time to of the kinetics. After the solution has briefly been stirred to homogenise the concentration of the enzyme the progressive development of a voltametric signal is observed which detaches itself from the residual current, producing a growth in the current in the zone of the potential expected for the oxidation of the free pADA, towards 0.17 V, as shown in FIG. 5. This Figure shows the variations in the current I as a function of the potential E in mV for different concentrations of free pADA.

The current produced is a result of the oxidation of the amine which has accumulated close to the surface (in the solution and probably, because of the not inconsiderable hydrophobicity of the pADA, in the SAM), during the time that separates two successive sweeps of potential, as shown in FIG. 6.

After several cycles of potential, the signal stabilises, indicating that the kinetics have come to an end. The gradient of the curve i-f(time) is proportional to the activity of the enzyme. The maximum intensity obtained is proportional to the quantity of substrate accessible to the enzyme on the surface, as shown in FIG. 7.

The method and device described above may be used to measure other enzymatic activities in a body fluid, notably enzymatic activities linked to the coagulation of whole blood.

1. Method of measuring an enzymatic activity in a body fluid, characterised in that it comprises the following steps:
a) providing a support having a conductive surface whereon is immobilised a substrate specific to the enzymatic activity to be measured, the substrate comprising an electroactive residue;
b) reacting a sample of the body fluid with the substrate so as to bring about an enzymatic reaction generating an electroactive product;
c) detecting the amount of electroactive product by amperometric measurement using the support in contact with the medium containing the enzyme.

2. Method according to claim 1 characterised in that the substrate is deposited in the form of at least one monomolecular layer of molecules corresponding to one of the following general formulae I and II:
S-Cₙ-(E)-P-X (I)
S-Cₙ-(-E)-X-P (II)
wherein:
S denotes a chemical group capable of reacting with the conductive surface,
Cₙ denotes an aliphatic methylene chain consisting of n carbon atoms,
E denotes a non-electroactive hydrophilic spacer of the polyethylene glycol type, which may optionally be present in order to complete the chain Cₙ,
P denotes a polypeptide sequence specific to the enzymatic activity which is to be measured, and
X denotes an electroactive residue capable of being oxidised or reduced within a potential range which is accessible in the medium, after the enzymatic hydrolysis.

3. Method according to claim 2, characterised in that in formulae I and II S denotes a thiol group, Cₙ denotes a hydrophobic carbon chain having n hydrocarbon members, particularly methylene, and carrying a hydrophilic spacer E, P denotes a peptide sequence with at least two amino acids of a substrate specific to the enzymatic activity which is to be measured, and X is an aromatic amide, particularly para-aminodiphenylamide.

4. Method according to one of claims l to 3, characterised in that the body fluid is a fluid which may be cloudy and/or coloured.

5. Method according to one of claims 1 to 4, characterised in that the body fluid is whole blood.

6. Method according to one of claims 1 to 5, characterised in that the enzymatic activity is that of a clotting factor or of complement from whole blood, particularly a clotting factor such as thrombin.

7. Method according to one of claims 1 to 5, characterised in that the enzymatic activity is used to titrate a endogenous or exogenous inhibitor or cofactor of blood coagulation.

8. Method according to one of claims 1 to 7, characterised in that in step b) one drop of the sample, particularly whole blood, is deposited directly on the support on which the substrate is immobilised.

9. Method according to one of claims 1 to 8, characterised in that the conductive surface is a layer of a metal capable of immobilising the covalent layers (I) or (II) particularly gold.

10. Method according to one of claims 1 to 9, characterised in that the detection step comprises using amperometric measuring electrodes comprising a working electrode, a counter-electrode and optionally a reference electrode.

11. Method according to claim 10, characterised in that the working electrode is made up of the support.

12. Method according to one of claims 10 and 11, characterised in that the counter-electrode and reference electrode are one and the same.

13. Method according to one of claims 1 to 12, characterised in that the detection step comprises measuring the amplitude of a signal which is proportional to the quantity of electroactive product detected during the enzymatic reaction.

14. Device for measuring an enzymatic activity in a body fluid, for carrying out the method according to one of claims 1 to 13, characterised in that it comprises:
a support having a conductive surface;
a substrate specific to the enzymatic activity which is to be measured, and comprising an electroactive residue, the substrate being immobilised on the conductive surface;
means for detection by electrochemical, particularly amperometric, measurement.

15. Device according to claim 14, characterised in that the substrate is deposited in the form of at least one monomolecular layer of molecules corresponding to one of the following general formulae I and II:
S-Cₙ-(E)-P-X (I)
S-Cₙ-(E)-X-P (II)
wherein:
S denotes a chemical group capable of reacting with the conductive surface
Cₙ denotes an aliphatic methylene chain consisting of n carbon atoms,
E denotes a non-electroactive hydrophilic spacer of the polyethylene glycol type, which may optionally be present in order to complete the chain Cₙ,
P denotes a polypeptide sequence specific to the enzymatic activity which is to be measured, and
X denotes an electroactive residue capable of being oxidised or reduced within a potential range which is accessible in the medium, after the enzymatic hydrolysis.

16. Device according to claim 15, characterised in that in formulae I and II S denotes a thiol group, C denotes a hydrophobic carbon chain having n hydrocarbon members, particularly methylene, and carrying a hydrophilic spacer E, P denotes a peptide sequence with at least two amino acids of a substrate specific to the enzymatic activity which is to be measured, and X is an aromatic amide, particularly para-aminodiphenylamide.

17. Device according to one of claims 14 to 16, characterised in that the conductive surface is a layer of a noble metal, particularly gold, deposited on the support.

18. Device according to one of claims 14 to 17, characterised in that the detection means comprise amperometric measuring electrodes comprising a working electrode, a counter-electrode and optionally a reference electrode.

19. Device according to claim 18, characterised in that the working electrode is made up of the support.

20. Device according to one of claims 18 and 19, characterised in that the counter-electrode and reference electrode are one and the same.

21. Device according to one of claims 18 to 20, characterised in that the amperometric detecting means comprise a potentiostat capable of delivering, between the working electrode and reference electrode, a difference in potential which is periodical and adjustable to the detection range of the marker.

22. Device according to one of claims 14 to 21, characterised in that it comprises a series of substrates which are specific to different enzymes, respectively thus allowing simultaneous analysis and establishment of a diagnostic profile.
